(19) United States
(12) Patent Application Publication
(10) Pub. No.: US 2008/0139614 A1
(43) Pub. Date: Jun. 12, 2008

(54) Treatment Of Stroke With Histamine H3 Inverse Agonists Or Histamine H3 Antagonists

(76) Inventors: Guy R. Seabrook, Blue Bell, PA (US); Ken S. Koblan, Carversville, PA (US); Tony Wei-Hsiu Ho, Berwyn, PA (US)

Correspondence Address: MERCK AND CO., INC, P O BOX 2000, RAHWAY, NJ, 07065-0907, US

(21) Appl. No.: 11/667,817

(22) PCT Filed: Nov. 18, 2005
(86) PCT No.: PCT/US2005/042365

§ 371 Date: May 14, 2007

Related U.S. Application Data
(60) Provisional application No. 60/630,513, filed on Nov. 23, 2004.

(51) Int. Cl. A61K 31/445; A61K 35/00; A61P 25/00

(52) U.S. Cl. 514/317; 514/789

(57) ABSTRACT

Histamine H3 inverse agonists or histamine H3 antagonists are useful, alone or in combination with an anti-stroke agent, for treating stroke.

### BACKGROUND OF THE INVENTION

Stroke is a cerebrovascular event, which occurs when the normal bloodflow to the brain is disrupted, and the brain receives too much or too little blood. Stroke is one of the leading causes of death worldwide, and is also one of the most common causes of neurologic disability.

Ischemic stroke, which is the most common type of stroke, results from insufficient cerebral circulation of blood caused by obstruction of the inflow of arterial blood. Normally, adequate cerebral blood supply is ensured by a system of arteries within the brain. However, various disorders, including inflammation and atherosclerosis, can cause a thrombus, i.e., a blood clot that forms in a blood vessel. The thrombus may interrupt arterial blood flow, causing brain ischemia and consequent neurologic symptoms. Ischemic stroke may also be caused by the lodging of an embolus (an air bubble) from the heart in an intracranial vessel, causing decreased perfusion pressure or increased blood viscosity with inadequate cerebral blood flow. An embolus may be caused by various disorders, including atrial fibrillation and atherosclerosis.

A second type of stroke, hemorrhagic stroke, involves a hemorrhage or rupture of an artery leading to the brain. Hemorrhagic stroke results in bleeding into brain tissue, including the epidural, subdural, or subarachnoid space of the brain. A hemorrhagic stroke typically results from the rupture of an arteriosclerotic vessel that has been exposed to arterial hypertension or to thrombosis.

During acute ischemic stroke, i.e., the period from the cerebrovascular event up to 24 hours after the event, the arterial occlusion results in an immediate infarcted core of brain tissue, where cerebral blood flow is significantly reduced, for example to less than 20% of the normal blood flow. The infarcted core suffers irreversible damage due to significant cell death. The length of time that ischemia persists, and the severity of the ischemia, contribute to the extent of injury. An area around the infracted core, known as the ischemic penumbra, suffers a delayed and less severe infarct. For example, during acute stroke the penumbra may have a reduction in blood flow of from about 20-40% of normal blood flow.

While not fully understood, the pathogenesis of ischemic stroke involves a complex cascade of multiple interacting biochemical events, which lead to acute neurologic injury and reduced neurological function. Ischemia results in the depletion of cellular energy stores of ATP, and the failure of sodium and potassium ion pumps. This leads to depolarization of neurons in the brain, and consequent excitotoxicity, i.e. excessive activity of excitatory amino acids, including glutamate, resulting in neuronal damage. In addition, the cascade leads to an increase in intracellular calcium. The presence of intracellular calcium in turn leads to the activation of intracellular enzymes and neuronal death. Lyden et al., J Stroke and Cerebrovasc Dis 2000; 9 (6, Suppl 2); 9-14. Excitotoxicity also results in the activation of enzymes, phospholipases, proteases, and nitric oxide synthases, and the production of oxygen free radicals. Each of these events contribute to the neuronal cell death of stroke. Nicotera et al, J Cerebr Blood Flow & Metab 19(6); 583-591 (1999).

One opportunity for pharmacologic intervention in stroke is the prevention or reduction of risk of stroke in patients at risk for stroke. There are many known risk factors for stroke, including vascular inflammation, atherosclerosis, arterial hypertension, diabetes, hyperlipidemia and atrial fibrillation. At risk patients have been treated with agents to control blood pressure or manage blood lipid level, and have been treated with antiplatelet agents (such as clopidrogel) and anticoagulants. Patients who have suffered myocardial infarction and are at risk for stroke are often treated with angiotensin-converting enzyme inhibitors (ACE inhibitors) or beta adrenergic antagonists (beta blockers).

A second opportunity for pharmacological treatment of stroke is the treatment of acute stroke. However, current pharmacologic therapies for treating acute stroke are limited to restoring blood flow within a narrow therapeutic time window of less than three hours after stroke. The only agents which have shown effectiveness in treating acute stroke are thrombolytics (such as rt-PA) and urokinase. There remains a need for agents which are effective within a longer therapeutic time window.

Another opportunity for pharmacological treatment of stroke is recovery or restoration after the acute stroke period, i.e. the reduction or prevention of secondary cell damage in the penumbra. Although some neuroprotective agents have demonstrated efficacy in preclinical animal models of stroke, favorable results have not always been duplicated in human clinical trials. There remains a need for agents which are effective in reducing or preventing secondary cell damage after stroke.

### SUMMARY OF THE INVENTION

The present invention is directed to the use of a histamine H3 inverse agonist or a histamine H3 antagonist, alone or in combination with an anti-stroke agent, for treating stroke.

### DESCRIPTION OF THE INVENTION

The present invention is directed to the use of a histamine H3 inverse agonist or a histamine H3 antagonist, or a pharmaceutically acceptable salt thereof, alone or in combination with an anti-stroke agent, for treating, ameliorating or controlling stroke or the neurologic injuries caused by stroke in a patient in need thereof.

An embodiment of the present invention is directed to a method for treating, ameliorating or controlling stroke or the neurologic injuries caused by stroke in a patient in need thereof that comprises administering to the patient a therapeutically effective amount of a histamine H3 inverse agonist or a histamine H3 antagonist, or a pharmaceutically acceptable salt thereof.

An embodiment of the present invention is directed to a method for treating, ameliorating or controlling hyperthermia caused by stroke in a patient in need thereof that comprises administering to the patient a therapeutically effective amount of a histamine H3 inverse agonist or a histamine H3 antagonist, or a pharmaceutically acceptable salt thereof.

An embodiment of the present invention is directed to a method for enhancing functional recovery following stroke in a patient in need thereof that comprises administering to the patient a therapeutically effective amount of a histamine H3 inverse agonist or histamine H3 antagonist, or a pharmaceutically acceptable salt thereof.

An embodiment of the present invention is directed to a method for reducing hospitalization following stroke in a patient in need thereof that comprises administering to the patient a therapeutically effective amount of a histamine H3 inverse agonist or histamine H3 antagonist, or a pharmaceutically acceptable salt thereof.

Although a histamine H3 inverse agonist or antagonist is useful alone for treating, ameliorating or controlling stroke or the neurologic injuries caused by stroke, it will be appreciated that a combination of another anti-stroke drug with a histamine H3 inverse agonist or antagonist may provide an enhanced effect in treating, ameliorating or controlling stroke or the neurologic injuries caused by stroke.

The present invention also provides a method for treating, ameliorating or controlling stroke or the neurologic injuries caused by stroke, which method comprises administration to a patient in need of such treatment of an amount of a histamine H3 inverse agonist or histamine H3 antagonist, or a pharmaceutically acceptable salt thereof and an amount of an anti-stroke agent, such that together they give effective relief.

As used herein, the term “stroke” refers to a clinical event involving impairment of cerebral circulation, that results in neurologic injury. Typically, stroke is manifest by the abrupt onset of a focal neurologic deficit. Stroke results from a rupture or obstruction (as by a thrombus or embolus) of an artery of the brain.

As used herein, the term “ischemic stroke” refers to stroke characterized by localized tissue anemia due to obstruction of the inflow of arterial blood. Ischemic stroke is usually caused by atherothrombosis or embolism of a major cerebral artery, but may also be caused by coagulation disorders or nonatheromatous vascular disease.

The subject or patient to whom a compound of the present invention is administered is generally a human being, male or female, in whom treatment of stroke is desired, but may also encompass other mammals, such as dogs, cats, mice, rats, cattle, horses, sheep, rabbits, monkeys, chimpanzees or other apes or primates, for which treatment of stroke is desired.

One class of patients to which a compound of the invention may be administered is a patient at risk for stroke. As used herein, the term “patient at risk for stroke” means an individual who has had a previous stroke, or has a risk factor for stroke. Known risk factors for stroke include atherosclerosis, arterial hypertension, lipohyalinosis, hyperlipidemia, hypercholesterolemia, atrial fibrillation, smoking, inflammatory markers (including C-reactive protein), infection, homocysteine, sleep-disordered breathing, cerebral autosomal dominant arteriopathy with subcortial infarcts and leuko-encephalopathy (CADASIL), migraine, sickle-cell anemia, antiphospholipid antibody syndrome, arterial dissection, cocaine abuse and obesity.

As used herein, the term “treatment” or “treating” means any administration of a compound of the present invention and includes (1) inhibiting stroke or the symptoms of stroke in an animal that is experiencing or displaying the pathology or symptomatology of stroke (i.e., arresting further development of the pathology and/or symptomatology), (2) ameliorating stroke or the symptoms of stroke in an animal that is experiencing or displaying the pathology or symptomatology of stroke (i.e., reversing the pathology and/or symptomatology), and (3) enhancing functional recovery following stroke or reducing hospitalization following stroke. The term “controlling” includes preventing, treating, eradicating, ameliorating or otherwise reducing the severity of stroke, or reducing the risk of stroke.

Efforts at “controlling” stroke (including preventing stroke) can be divided into the primary prevention of stroke (treatment of patients who have not had any prior transient ischemic attacks of strokes, and have no neurological symptoms) and secondary prevention of stroke (treatment of patients who have had a prior transient ischemic attack or stroke). Primary prevention of stroke includes non-pharmacologic interventions, such as smoking cessation, healthy eating patterns, increased physical activity and weight management. Primary prevention also includes certain pharmacologic interventions, such as blood pressure control, treatment of atrial fibrillation, and management of diabetes, if appropriate. As part of the primary prevention of stroke, patients at high risk of coronary heart disease are often treated with aspirin. As part of primary prevention, patients having high amounts of low density lipoprotein (LDL) are often subject to blood lipid management, to reduce LDL levels to acceptable levels, e.g. below 160 g/dl.

The secondary prevention of stroke often involves the same pharmacologic and non-pharmacologic interventions used for primary prevention, including blood pressure control, treatment of atrial fibrillation, management of diabetes, treatment with aspirin, and blood lipid management. Additional common secondary prevention interventions include the use of antiplatelet agents (such as clopidrogel), anticoagulants (such as warfarin), and anti-hypertension agents (such as beta andrenergic antagonists).

A second class of patients to which a compound of the invention may be administered are acute stroke patients, i.e., patients who have suffered ischemic stroke within the last 7 days. One preferred class of acute stroke patients are those who have suffered stroke within the last 3 days. A more preferred class of acute stroke patients are those who have suffered stroke within the last 48 hours, even more preferably within the last 24 hours. As common in the art of treating stroke, patients may be classified according to the period of time when stroke occurred. So, for example, one class of acute stroke patients are those who have suffered stroke within the last 18 hours. Another class of acute stroke patients are those who have suffered stroke within the last 12 hours. Another class of acute stroke patients are those who have suffered stroke within the last 8 hours. Another class of acute stroke patients are those who have suffered stroke within the last 6 hours. Another class of acute stroke patients are those who have suffered stroke within the last 4 hours. Another class of acute stroke patients are those who have suffered stroke within the last 3 hours.

Treatment of acute stroke, i.e. treatment during the cerebral event causing stroke and the 7 days thereafter, involve treatment with thrombolytics such as recombinant tissue plasminogen activator (rtPA). However, rtPA has only been approved for treatment of acute stroke for use within the first three hours after stroke. Another potential agent for treatment of acute stroke is the neuroprotectant edaravone, which has been approved in Japan.

During acute ischemic stroke, the arterial occlusion caused by the thrombus or embolus results in an immediate infarcted core of brain tissue, where cerebral blood flow is significantly reduced, for example to less than 20% of the normal blood flow. The infarcted core suffers irreversible damage due to significant cell death. The length of time that ischemia persists, and the severity of the ischemia, contribute to the extent of the infarct. An area around the infracted core, known as the ischemic penumbra, suffers a delayed and less severe infarct. For example, during acute stroke the penumbra may have a reduction in blood flow of from about 20-40%.

Patients who have suffered stroke more than 24 hours previously often develop cerebral edema which typically occurs at from one to five days after stroke. As used herein, the term “cerebral edema” refers to fluid collecting in brain tissue due to cellular swelling and the breakdown of the blood-brain barrier. Post-stroke cerebral edema may also involve the exuding of cerebrospinal fluid from ependymal lining, or the creation of an osmotic environment due to blood clots or tissue injury. The osmotic environment allows the movement of water into interstitial spaces. Post-stroke cerebral edema is often responsible for a worsening in the stroke patient's clinical status. Stroke may also result in hyperthermia in a patient.

A third class of patients to which a compound of the present invention may be administered are patients who have suffered stroke more than 7 days previously, who are typically in need of restorative treatment.

In an embodiment of the present invention the histamine H3 inverse agonist is a selective histamine H3 inverse agonist. In an embodiment of the present invention the histamine H3 inverse agonist possesses a selectivity for the histamine H3 receptor relative to all other non-histamine G-protein coupled receptors of at least 5 fold as measured by the ratio of IC₅₀ for the histamine H3 receptor to the IC₅₀ for each of the other non-histamine G-protein coupled receptors. In another embodiment of the present invention the histamine H3 receptor inverse agonist possesses a selectivity for the histamine H3 receptor relative to all other non-histamine G-protein coupled receptors of at least 50 fold as measured by the ratio of IC₅₀ for the histamine H3 receptor to the IC₅₀ for each of the non-histamine G-protein coupled receptors. In another embodiment of the present invention the histamine H3 receptor inverse agonist possesses a selectivity for the histamine H3 receptor relative to all other non-histamine G-protein coupled receptors of at least 100 fold as measured by the ratio of IC₅₀ for the histamine H3 receptor to the IC₅₀ for each of the non-histamine G-protein coupled receptors. In another embodiment of the present invention the histamine H3 receptor inverse agonist possesses a selectivity for the histamine H3 receptor relative to other non-histamine G-protein coupled receptors of at least 200 fold as measured by the ratio of IC₅₀ for the histamine H3 receptor to the IC₅₀ for each of the other non-histamine G-protein coupled receptors. In an embodiment of the present invention the histamine H3 inverse agonist possesses a selectivity for the histamine H3 receptor relative to all other histamine receptors of at least 5 fold as measured by the ratio of IC₅₀ for the histamine H3 receptor to the IC₅₀ for each of the other histamine receptors. In another embodiment of the present invention the histamine H3 receptor inverse agonist possesses a selectivity for the histamine H3 receptor relative to all other histamine receptors of at least 50 fold as measured by the ratio of IC₅₀ for the histamine H3 receptor to the IC₅₀ for each of the other histamine receptors. In another embodiment of the present invention the histamine H3 receptor inverse agonist possesses a selectivity for the histamine H3 receptor relative to all other histamine receptors of at least 100 fold as measured by the ratio of IC₅₀ for the histamine H3 receptor to the IC₅₀ for each of the other histamine receptors. In another embodiment of the present invention the histamine H3 receptor inverse agonist possesses a selectivity for the histamine H3 receptor relative to the other histamine receptors of at least 200 fold as measured by the ratio of IC₅₀ for the histamine H3 receptor to the IC₅₀ for each of the other histamine receptors.

In an embodiment of the present invention the histamine H3 antagonist is a selective histamine H3 antagonist. In an embodiment of the present invention the histamine H3 antagonist possesses a selectivity for the histamine H3 receptor relative to all other non-histamine G-protein coupled receptors of at least 5 fold as measured by the ratio of IC₅₀ for the histamine H3 receptor to the IC₅₀ for each of the other non-histamine G-protein coupled receptors. In another embodiment of the present invention the histamine H3 receptor antagonist possesses a selectivity for the histamine H3 receptor relative to all other non-histamine G-protein coupled receptors of at least 50 fold as measured by the ratio of IC₅₀for the histamine H3 receptor to the IC₅₀ for each of the non-histamine G-protein coupled receptors. In another embodiment of the present invention the histamine H3 receptor antagonist possesses a selectivity for the histamine H3 receptor relative to all other non-histamine G-protein coupled receptors of at least 100 fold as measured by the ratio of IC₅₀ for the histamine H3 receptor to the IC₅₀ for each of the non-histamine G-protein coupled receptors. In another embodiment of the present invention the histamine H3 receptor antagonist possesses a selectivity for the histamine H3 receptor relative to other non-histamine G-protein coupled receptors of at least 200 fold as measured by the ratio of IC₅₀ for the histamine H3 receptor to the IC₅₀ for each of the other non-histamine G-protein coupled receptors. In an embodiment of the present invention the histamine H3 antagonist possesses a selectivity for the histamine H3 receptor relative to all other histamine receptors of at least 5 fold as measured by the ratio of IC₅₀for the histamine H3 receptor to the IC₅₀ for each of the other histamine receptors. In another embodiment of the present invention the histamine H3 receptor antagonist possesses a selectivity for the histamine H3 receptor relative to all other histamine receptors of at least 50 fold as measured by the ratio of IC₅₀ for the histamine H3 receptor to the IC₅₀ for each of the other histamine receptors. In another embodiment of the present invention the histamine H3 receptor antagonist possesses a selectivity for the histamine H3 receptor relative to all other histamine receptors of at least 100 fold as measured by the ratio of IC₅₀ for the histamine H3 receptor to the IC₅₀ for each of the other histamine receptors. In another embodiment of the present invention the histamine H3 receptor antagonist possesses a selectivity for the histamine H3 receptor relative to the other histamine receptors of at least 200 fold as measured by the ratio of IC₅₀ for the histamine H3 receptor to the IC₅₀ for each of the other histamine receptors.

In the present invention, a histamine H3 inverse agonist or antagonist may be employed as the free base or as a pharmaceutically acceptable salt thereof. Representative histamine H3 receptor ligands and pharmaceutically acceptable salts thereof are disclosed in e.g., U.S. Pat. Nos. 5,486,526; 5,652,258; 5,990,317; 6,008,240; 6,437,147 and PCT Patent Publications WO 96/40126; WO 96/38142; WO 01/300346; WO 01/068651; WO 01/068652; WO 02/015905; WO 03/004480; WO 03/024928; WO 03/066604; WO 03/0236259; and may be prepared by methods described therein. Representative histamine H3 inverse agonists include: 4-{(1R,2R)-trans-2-[O-(2-cyclohexylethyl)carboxamido]-cyclopropyl}imidazole, 4-{(IR,2R)-trans-2-[O-(2-cyclohexylmethyl)carboxamido]cyclopropyl}-imidazole; 3-(1H-imidazol-4 yl)propyl-di(p-fluorophenyl)-methyl ether; and 2-(1-cyclopentylpiperidine-4-yloxy)-5-(4-cyanophenyl)pyrimidine.

The identification of a compound as a histamine H3 inverse agonist or a histamine H3 antagonist may be readily determined without undue experimentation by methodology well known in the art. The efficacy of a histamine H3 inverse agonist or a histamine H3 antagonist in treating stroke may be readily determined without undue experimentation by methodology well known in the art, for example the harmaline-induced tremor model in rats, Sinton, et al., Pflugers Archive Eur. J. Phys., 414(1) 31-36 (1989). In this model, a histamine H3 inverse agonist exhibited a dose dependent ability to decrease harmaline-induced tremor. In particular, at a dose of 1 mg/kg the histamine H3 inverse agonist 2-(1-cyclopentylpiperidine-4-yloxy)-5-(4-cyanophenyl)pyrimidine exhibited an 8.4% reversal of harmaline-induced tremor; at a dose of 3 mg/kg the histamine H3 inverse agonist exhibited an 63.3% reversal of harmaline-induced tremor; and at a dose of 10 mg/kg the histamine H3 inverse agonist exhibited an 84.4% reversal of harmaline-induced tremor. The ability of the histamine H3 inverse agonist or histamine H3 antagonist to be used in the present invention to treat stroke may be determined by these methods.

2-(1-Cyclopentylpiperidine-4-yloxy)-5-(4-cyanophenyl) pyrimidine: To a DMF solution (10 mL) of 2-chrolo-5-bromo-pyrimidine (300 mg, 1.56 mmol), 1-tert-butoxy carbonyl-4-hydroxy-piperidine (408 g, 2.03 mmol) and cesium carbonate were added. The reaction mixture was stirred for 14 hours at room temperature. Water was added to a reaction mixture and the reaction mixture was extracted with ethyl acetate. The organic layer was washed with brine, dried over anhydrous sodium sulfate and concentrated in vacuo. The residue was purified by silica gel column chromatography (C-300, hexane:ethyl acetate=10:1) to afford 2-(1-tert-butoxycarbonylpiperidine-4-yloxy)-5-bromopyrimidine. To a 2-(1-tert-butoxycarbonylpiperidine-4-yloxy)-5-(4-cyanophenyl) pyrimidine (149 mg, 0.42 mol), 2-dimethoxyethane (2.0 mL) and 2N sodium carbonate(0.7 mL) were added, and then 4-cyano-boric acid (75.2 mg, 0.51 mmol) and tetrakis(triphenyl phosphine)palladium(0) (10 mg, 0.0087 mmol) were added. The reaction mixture was stirred at 90 degree for 30 hours under N₂ atmosphere. After the reaction mixture was cooled to room temperature, and extracted with ethyl acetate. The organic layer was washed with brine, dried over anhydrous sodium sulfate, and concentrated in vacuo. The residue was purified by silica gel column chromatography (C-300, hexane:ethyl acetate=3:1) to afford 2-(1-tert-butoxycarbonylpiperidine-4-yloxy)-5-(4-cyanophenyl)pyrimidine. To a methylene chloride solution of 2-(1-tert-butoxycarbonylpiperidine-4-yloxy)-5-(4-cyanophenyl) pyrimidine (122 mg, 0.32 mmol) was added trifluoroacetic acid (1.5 ml) and the reaction mixture was stirred at room temperature for 2.5 hours. The reaction mixture was concentrated in vacuo and the residue was extracted with chloroform. The organic layer was sodium hydrogen carbonate, brine, dried over anhydrous sulfate and concentrated in vacuo to afford 2-(piperidine-4-yloxy)-5-(4-cyanophenyl)-pyrimidine. To a methanol solution (3.0 mL) of 2-(pyperidine-4-yloxy)-5-(4-cyanophenyl)pyrimidine (46 mg, 0.16 mmol), cyclopentanone and 0.3N zinc chloride-sodium cyanoborate solution(0.55 mL) were added and the reaction mixture was stirred at room temperature for 14 hours. The reaction mixture was concentrated in vacuo and the residue was extracted with chloroform. The organic layer was washed with brine, dried over anhydrous sodium sulfate and concentrated in vacuo. The residue obtained was purified by silica gel column chromatography (eluted with chloroform:methanol=10:1) to afford 2-(1-cyclopentylpiperidine-4-yloxy)-5-(4-cyanophenyl) pyrimidine. ¹H NMR (300 MHz, CDCl₃, δppm): 1.38-1.78 (6H, m), 1.82-2.04 (4H, m), 2.08-2.21 (2H, m), 2.32-2.63 (3H, m), 2.74-2.96 (2H, m), 5.07-5.18 (1H, m), 7.62(2H, d, J=8.6 Hz), 7.78 (1H, d, J=8.6 Hz),8.73 (2H, s), Mass (ESI): 349 (M+H).

Accordingly, the present invention includes within its scope the use of a histamine H3 inverse agonist or antagonist, alone or in combination with other agents, for the subject indications in a warm-blooded animal. For the purposes of this disclosure, a warm-blooded animal is a member of the animal kingdom which includes but is not limited to mammals and birds. The preferred mammal for purposes of this invention is human.

The subject treated in the present methods is generally a mammal, preferably a human, male or female. In the present invention, it is preferred that the subject mammal is a human. Although the present invention is applicable both old and young people, in certain aspects such as cognition enhancement it would find greater application in elderly people. The term “therapeutically effective amount” means the amount of the subject compound that will elicit the biological or medical response of a tissue, system, animal or human that is being sought by the researcher, veterinarian, medical doctor or other clinician.

This particular application of a histamine H3 inverse agonist or antagonist provides unexpected benefit relative to the administration of other agents for the subject indications. For example, a histamine H3 inverse agonist or antagonist may exhibit a rapid onset of action and a reduced side-effect profile relative to other agents used for the treatment of stroke.

The term “composition” as used herein is intended to encompass a product comprising the specified ingredients in the specified amounts, as well as any product which results, directly or indirectly, from combination of the specified ingredients in the specified amounts. Such term in relation to pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof.

The terms “administration of” or “administering a” compound should be understood to mean providing a compound of the invention or a prodrug of a compound of the invention to the individual in need of treatment in a form that can be introduced into that individual's body in a therapeutically useful form and therapeutically useful amount.

The terms “effective amount” or “therapeutically effective amount” means the amount of the subject compound that will elicit the biological or medical response of a tissue, system, animal or human that is being sought by the researcher, veterinarian, medical doctor or other clinician. As used herein, the term “treatment” refers to the treatment of stroke, particularly in a patient who demonstrates symptoms of stroke.

As used herein, the term “prodrug” refers to a molecule that is inert, i.e. not pharmacologically active, but that has pharmacological activity upon activation by a biological system. For example, a prodrug is a compound which is inert when in a tablet, capsule or other pharmaceutical composition, but is modified and becomes pharmacologically active in vivo, upon ingestion by a mammal.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts in the solid form may exist in more than one crystal structure, and may also be in the form of hydrates. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylene-diamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, trifluoroacetic acid and the like. Particularly preferred are citric, hydrobromic, hydrochloric, maleic, phosphoric, sulfuric, fumaric, trifluoroacetic and tartaric acids.

The compounds employed in the present invention, may have chiral centers and occur as racemates, racemic mixtures and as individual diastereomers, or enantiomers with all isomeric forms being included in the present invention. Therefore, where a compound is chiral, the separate enantiomers, substantially free of the other, are included within the scope of the invention; further included are all mixtures of the two enantiomers.

A histamine H3 inverse agonist or antagonist may be used alone or in combination with other agents or with other compounds which are known to be beneficial in the subject indications. A histamine H3 inverse agonist or antagonist and the other agent may be co-administered, either in concomitant therapy or in a fixed combination. For example, a histamine H3 inverse agonist or antagonist may be administered in conjunction with other compounds which are known in the art for the subject indications. It will be appreciated that when using a combination of the present invention, a histamine H3 inverse agonist or antagonist and the other agent may be in the same pharmaceutically acceptable carrier and therefore administered simultaneously. They may be in separate pharmaceutical carriers such as conventional oral dosage forms which are taken simultaneously. The term “combination” also refers to the case where the compounds are provided in separate dosage forms and are administered sequentially. Therefore, by way of example, the anti-stroke agent may be administered as a tablet and then, within a reasonable period of time, a histamine H3 inverse agonist or antagonist may be administered either as an oral dosage form such as a tablet or a fast-dissolving oral dosage form. By a “fast-dissolving oral formulation” is meant, an oral delivery form which when placed on the tongue of a patient, dissolves within about 10 seconds.

Suitable anti-stroke agents of use in combination with a histamine H3 inverse agonist or antagonist include a tissue plasminogen activator (tPA), a COX-2 inhibitor, a nitric oxide synthase inhibitor, a Rho kinase inhibitor, an angiotension II type-1 receptor antagonist, a glycogen synthase kinase 3 inhibitor, a sodium channel blocker, a calcium channel blocker, a p38 MAP kinase inhibitor, a thromboxane AX-synthetase inhibitor, a statin (an HMG CoA reductase inhibitor), a neuroprotectant (including an antioxidant, a reactive astrocyte inhibitor, an NMDA receptor antagonist, such as analogs of ifenprodil, an NR2B antagonist, a free radical-trapping, such as disufenton, a 5-HT1A agonist, such as repinotan), edaravone, a GSK-3beta inhibitor, a beta andrenergic blocker, an NMDA receptor antagonist, a platelet fibrinogen receptor antagonist, a thrombin inhibitor, an antihypertensive agent or a vasodilator, or a pharmaceutically acceptable salt thereof.

The present invention includes within its scope a pharmaceutical composition for the subject indications comprising, as an active ingredient, a histamine H3 inverse agonist or antagonist in association with a pharmaceutical carrier or diluent. Optionally, the active ingredient of the pharmaceutical compositions can comprise another agent in addition to a histamine H3 inverse agonist or antagonist to minimize the side effects or with other pharmaceutically active materials wherein the combination enhances efficacy and minimizes side effects.

The present invention is further directed to a method for the manufacture of a medicament for the subject indications in humans comprising combining a compound that is a histamine H3 inverse agonist or antagonist with a pharmaceutical carrier or diluent.

It will be known to those skilled in the art that there are some compounds now being used for stroke. Combinations of these therapeutic agents some of which have also been mentioned herein with a histamine H3 inverse agonist or antagonist will bring additional, complementary, and often synergistic properties to enhance the desirable properties of these various therapeutic agents. In these combinations, a histamine H3 inverse agonist or antagonist and the therapeutic agents may be independently present in dose ranges from one one-hundredth to one times the dose levels which are effective when these compounds and secretagogues are used singly.

To illustrate these combinations, a histamine H3 inverse agonist or antagonist effective clinically at a given daily dose range may be effectively combined, at levels which are equal or less than the daily dose range, with such compounds at the indicated per day dose range. Typically, the individual daily dosages for these combinations may range from about one-fifth of the minimally recommended clinical dosages to the maximum recommended levels for the entities when they are given singly. It will be readily apparent to one skilled in the art that a histamine H3 inverse agonist or antagonist may be employed with other agents for the purposes of the present invention.

Naturally, these dose ranges may be adjusted on a unit basis as necessary to permit divided daily dosage and, as noted above, the dose will vary depending on the nature and severity of the disease, weight of patient, special diets and other factors.

These combinations may be formulated into pharmaceutical compositions as known in the art and as discussed below. A histamine H3 inverse agonist or antagonist may be administered alone or in combination by oral, parenteral (e.g., intramuscular, intraperitoneal, intravenous or subcutaneous injection, or implant), nasal, vaginal, rectal, sublingual, or topical routes of administration and can be formulated in dosage forms appropriate for each route of administration.

Solid dosage forms for oral administration include capsules, tablets, pills, powders and granules. In such solid dosage forms, the active compound is admixed with at least one inert pharmaceutically acceptable carrier such as sucrose, lactose, or starch. Such dosage forms can also comprise, as is normal practice, additional substances other than inert diluents, e.g., lubricating agents such as magnesium stearate. Illustrative of the adjuvants which may be incorporated in tablets, capsules and the like are the following: a binder such as gum tragacanth, acacia, corn starch or gelatin; an excipient such as microcrystalline cellulose; a disintegrating agent such as corn starch, pregelatinized starch, alginic acid and the like; a lubricant such as magnesium stearate; a sweetening agent such as sucrose, lactose or saccharin; a flavoring agent such as peppermint, oil of wintergreen or cherry. In the case of capsules, tablets and pills, the dosage forms may also comprise buffering agents. When the unit dosage form is a capsule, it may contain, in addition to materials of the above type, a liquid carrier such as fatty oil. Various other materials may be present as coatings or to otherwise modify the physical form of the dosage unit. Tablets and pills can additionally be prepared with enteric coatings and tablets may be coated with shellac, sugar or both.

Pharmaceutical compositions of the present compounds may be in the form of a sterile injectable aqueous or oleagenous suspension. The compounds of the present invention may also be administered in the form of suppositories for rectal administration. For topical use, creams, ointments, jellies, solutions or suspensions, etc., containing the compounds of the present invention may be employed. The compounds of the present invention may also be formulated for administered by inhalation. The compounds of the present invention may also be administered by a transdermal patch by methods known in the art. Compositions for nasal or sublingual administration are also prepared with standard excipients well known in the art.

It will be appreciated that the amount of a histamine H3 inverse agonist or antagonist will vary not only with the compositions selected but also with the route of administration, the nature of the condition being treated, and the age and condition of the patient, and will ultimately be at the discretion of the patient's physician or pharmacist.

The dosage of active ingredient in the compositions of this invention may be varied, however, it is necessary that the amount of the active ingredient be such that a suitable dosage form is obtained. The active ingredient may be administered to patients (animals and human) in need of such treatment in dosages that will provide optimal pharmaceutical efficacy. The selected dosage depends upon the desired therapeutic effect, on the route of administration, and on the duration of the treatment. The dose will vary from patient to patient depending upon the nature and severity of disease, the patient's weight, special diets then being followed by a patient, concurrent medication, and other factors which those skilled in the art will recognize. Generally, dosage levels of between 0.0001 to 10 mg/kg. of body weight daily are administered to the patient, e.g., humans and elderly humans. The dosage range will generally be about 0.5 mg to 1.0 g. per patient per day which may be administered in single or multiple doses. Preferably, the dosage range will be about 0.5 mg to 500 mg per patient per day; more preferably about 0.5 mg to 200 mg per patient per day; and even more preferably about 5 mg to 50 mg per patient per day. Specific dosages for administration include 10 mg, 30 mg and 60 mg.

Pharmaceutical compositions of the present invention may be provided in a solid dosage formulation preferably comprising about 0.5 mg to 500 mg active ingredient, more preferably comprising about 1 mg to 250 mg active ingredient. The pharmaceutical composition is preferably provided in a solid dosage formulation comprising about 1 mg, 5 mg, 10 mg, 25 mg, 50 mg, 100 mg, 200 mg or 250 mg active ingredient.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, effective dosages other than the particular dosages as set forth herein above may be applicable as a consequence of variations in the responsiveness of the mammal being treated for any of the indications with the compounds of the invention indicated above. Likewise, the specific pharmacological responses observed may vary according to and depending upon the particular active compounds selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

1-11. (canceled)

12. A method for treating, ameliorating or controlling stroke in a patient in need thereof that comprises administering to the patient a therapeutically effective amount of a histamine H3 inverse agonist or a histamine H3 antagonist, or a pharmaceutically acceptable salt thereof.

13. A method for treating, ameliorating or controlling a neurologic injury caused by stroke in a patient in need thereof that comprises administering to the patient a therapeutically effective amount of a histamine H3 inverse agonist or a histamine H3 antagonist, or a pharmaceutically acceptable salt thereof.

14. A method for enhancing functional recovery following stroke in a patient in need thereof that comprises administering to the patient a therapeutically effective amount of a histamine H3 inverse agonist or histamine H3 antagonist, or a pharmaceutically acceptable salt thereof.

15. The method of claim 12 wherein the method comprises administering a histamine H3 inverse agonist.

16. The method of claim 15 wherein the histamine H3 inverse agonist is a selective inverse agonist of the histamine H3 receptor.

17. The method of claim 12 wherein the method comprises administering a histamine H3 antagonist.

18. The method of claim 17 wherein the histamine H3 antagonist is a selective antagonist of the histamine H3 receptor.

19. The method of claim 12 wherein the a histamine H3 inverse agonist or histamine H3 antagonist, or a pharmaceutically acceptable salt thereof, is administered in conjunction with an anti-stroke agent, such that together they give effective relief.

20. The method of claim 19 wherein the histamine H3 inverse agonist or the histamine H3 antagonist, or a pharmaceutically acceptable salt thereof, is administered in combination with an agent selected from the group consisting of: a tissue plasminogen activator (tPA), a nitric oxide synthase inhibitor, a Rho kinase inhibitor, an angiotension II type-1 receptor antagonist, a glycogen synthase kinase 3 inhibitor, a sodium channel blocker, a calcium channel blocker, a p38 MAP kinase inhibitor, a thromboxane AX-synthetase inhibitor, an HMG CoA reductase inhibitor, a neuroprotectant, an antioxidant, a reactive astrocyte inhibitor, an NMDA receptor antagonist, analogs of ifenprodil, an NR2B antagonist, a free radical-trapping, disufenton, a 5-HT1A agonist, repinotan, edaravone, a GSK-3beta inhibitor, a beta andrenergic blocker, an NMDA receptor antagonist, a platelet fibrinogen receptor antagonist, a thrombin inhibitor, an antihypertensive agent, a vasodilator, or a pharmaceutically acceptable salt thereof.
